(19) United States
(12) Patent Application Publication
(10) Pub. No.: US 2007/0088213 A1
(43) Pub. Date: Apr. 19, 2007

(54) Ultrasonic diagnostic imaging with automatic adjustment of beamforming parameters

(75) Inventor: Mckeen Dunn Poland, Andover, MA (US)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS N.V., EINDHOVEN (NL)

(21) Appl. No.: 10/578,979

(22) PCT Filed: Nov. 17, 2004
(86) PCT No.: PCT/IB04/52466

§ 371 Date: May 11, 2006

Related U.S. Application Data
(60) Provisional application No. 60/523,815, filed on Nov. 20, 2003.

(51) Int. Cl. A61B 8/00

(52) U.S. Cl. 600/437

(57) ABSTRACT

An ultrasound imaging system and method is provided for acquiring a series of images. The ultrasound system includes a transducer (11) for emitting and receiving ultrasound energy, a beamformer assembly (402) for beamforming in accordance with at least one beamforming parameter the emitted and received ultrasound energy for generating a plurality of scan lines and acquiring a series of images, and at least one user input device (460) for enabling a user to select at least one automatic adjustment parameter includin g an axis selectable from a line in space for which scan lines of a set of scan lines generated during one of rotation about the line in space or translation along the line in space lie within an acoustic field of view of the transducer and an adjustment factor. The ultrasound system further includes a control unit (412) for controlling the beamformer assembly during acquisition of the series of images for adjusting the at least one beamformer parameter for an image being acquired with respect to a previous image acquired in accordance with the at least one automatic adjustment parameter, for at least one of rotating about the axis and translating along the axis the image being acquired with respect to the previous image by an amount defined by the adjustment factor.

This invention relates to ultrasonic diagnostic imaging, and more particularly, to ultrasound systems that enable automatic adjustment of beamforming parameters.

In general, an ultrasound system emits pulses over a plurality of paths and converts echoes received from objects on the plurality of paths into electrical signals used to generate ultrasound data from which an ultrasound image can be displayed.

Ultrasound transducer assemblies comprise transducer elements, typically with damping and matching materials, that when excited by an electrical pulse emit ultrasound pulses and receive echoes. Transducer assemblies are packaged with associated electronics and connections in a housing that facilitates examination. Taken as a whole, such a combination (the transducer assembly, electronics, connections, and housing) is typically referred to as an ultrasound probe (or simply just “probe”). Probe classifications include 1-D probes (having a one-dimensional array of elements) or 2-D probes (having a two-dimensional array of elements).

In a linear phased array, all (or almost all) the elements are excited by a single pulse, but with small (typically less than 1 microsecond) time differences (“phasing”) between adjacent elements, so that the resulting sound pulses pile up along a specific direction (termed “steering”) for producing a plurality of beams or scan lines. In addition to steering the scan lines, the phased array can focus the scan lines, along the depth direction, by putting curvature in the phase delay pattern. More curvature places the focus closer to the transducer array, while less curvature moves the focus deeper. Upon reception of echoes, delays are used to time the sampling of the raw points of data from which ultrasound image data is produced. Focal points of the plurality of scan lines generated per pulse lie on a predetermined geometric shape, such as a planar surface, a curved surface or a frusto-conical surface. The orientation and shape of the geometric shape is determined by the steering and focusing of the scan lines.

The apparatus that creates the various delays is called a beamformer. Known beamformers have traditionally operated in the analog domain employing expensive circuits capable of delivering a new point of data (dynamically delayed) every nano-second. More recently, digital beamformers have been developed, that provide delay by buffering A/D converted transducer output in a digital memory and varying the read times therefrom. Furthermore, the advent of micro-beamforming, which partitions data for reducing processing loads and provides for at least a portion of beamforming processing to be performed within the probe, reduces the time and circuitry used for transmission and processing of beamformed signals, which leaves resources available for more complicated steering processes.

Commercially available ultrasound systems may acquire first and second images, where the second image is acquired in a selected scan plane orthogonal to the plane of the first image, or rotated a selected amount relative to the plane of the first image. However, selectability of the scan plane is typically limited, and is relative to the plane of the first image. Furthermore, several iterations of user selection of a scan plane may be required in order to be able to select a desired scan plane. Moreover, each iteration is dependent upon user selection and activation.

Other commercially available ultrasound systems acquire virtually simultaneously a series of real-time 2-D images, where successive images are automatically acquired by rotating an imaging plane about an axis selected from one of the X, Y, and Z axes, where the X and Y axes define the plane of the face of the transducer. The series of 2-D images are acquired at low resolution and high speed for achieving the real-time affect, and high resolution is typically not achievable. The rotated images may be further processed on. the fly or at a designated time for generating a rendered 3D image. However, limitations exist for user selection of an axis of rotation, rate of image acquisition, a trigger for activation of acquisition of the successive images, location of an apex for the axis of rotation, range of rotation, scan line density, angle adjustment for each rotation, and resolution of the images to be acquired.

The present system provides an ultrasound imaging system for acquiring a series of images. The ultrasound system includes a transducer for emitting and receiving ultrasound energy, a beamformer assembly for beamforming in accordance with at least one beamforming parameter the emitted and received ultrasound energy for generating a plurality of scan lines and acquiring a series of images, and at least one user input device for enabling a user to select at least one automatic adjustment parameter including an axis selectable from a line in space for which scan lines of a set of scan lines generated during one of rotation about the line in space or translation along the line in space lie within an acoustic field of view of the transducer, and an adjustment factor. The ultrasound imaging system further includes a control unit for controlling the beamformer assembly during acquisition of the series of images for adjusting the at least one beamformer parameter for an image being acquired with respect to a previous image acquired in accordance with the at least one automatic adjustment parameter, for at least one of rotating about the axis and translating along the axis the image being acquired with respect to the previous image by an amount defined by the adjustment factor.

In another embodiment, the ultrasound imaging system includes a transducer for emitting and receiving ultrasound energy, a beamformer assembly for beamforming in accordance with at least one beamforming parameter the emitted and received ultrasound energy for acquiring a series of image, and circuitry for receiving a plurality of trigger signals driven by the occurrence of an event including at least one trigger signal driven by at least an asynchronous event. The ultrasound imaging system further includes a control unit for controlling the beamformer assembly during acquisition of the series of images for providing for acquiring individual images of the series of images in accordance with receipt of a respective at least one trigger signal of the at least one trigger signal, wherein the controlling includes adjusting the at least one beamformer parameter for an image acquisition with respect to a previous image acquisition in accordance with at least one predetermined automatic adjustment parameter, wherein the adjusting includes providing for shifting positioning of the image being acquired with respect to the previous image acquisition.

In another embodiment of the invention a method is provided for ultrasonically imaging a region of interest of a body with a transducer array generating a plurality of scan lines. The method includes the step of providing for receiving at least one automatic adjustment parameter including an axis selectable from a line in space for which scan lines of a set of scan lines generated during one of rotation about the line in space or translation along the line in space lie within an acoustic field of view of the transducer and an adjustment factor. The method further provides the step of providing for controlling beamforming of at least one of ultrasound energy emitted by the transducer and ultrasound energy received by the transducer in accordance with at least one beamforming parameter for acquiring a series of images by adjusting the at least one beamformer parameter for an image being acquired with respect to a previous image acquired in accordance with the at least one automatic adjustment parameter for at least one of rotating about the axis and translating along the axis the image being acquired with respect to the previous image by an amount defined by the adjustment factor.

In still another embodiment of the invention, the method for ultrasonically imaging include the steps of providing for receiving at last one automatic adjustment parameter and providing for receiving a plurality of trigger signals driven by an event including at least one trigger signal driven by at least an asynchronous event. The method further includes the step of providing for controlling beamforming of at least one of ultrasound energy emitted by the transducer and ultrasound energy received by the transducer in accordance with at least one beamforming parameter for acquiring a series of images, where respective images of the series of images are acquired in response to receipt of a respective at least one trigger signal of the at least one trigger signal, including adjusting the at least one beamformer parameter for an image acquisition with respect to a previous image acquisition in accordance with the at least one automatic adjustment parameter, wherein the adjusting includes providing for shifting positioning of the image being acquired with respect to the previous image acquisition.

Various embodiments of the invention will be described herein below with reference to the figures wherein:

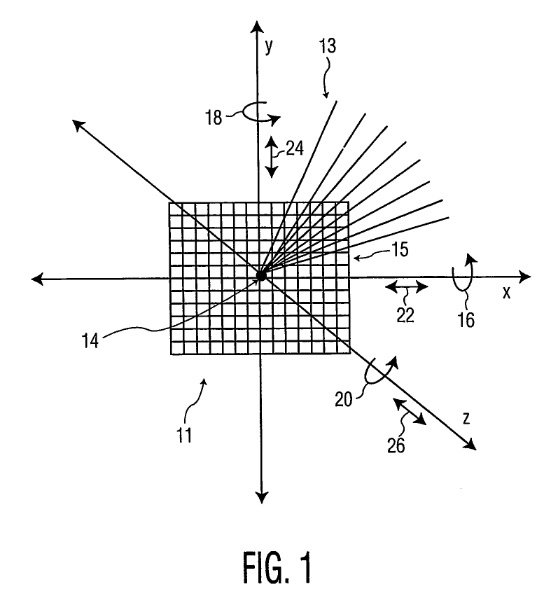

FIG. 1 is a schematic diagram, of a 2-D transducer array and reference axes in accordance with the present invention;

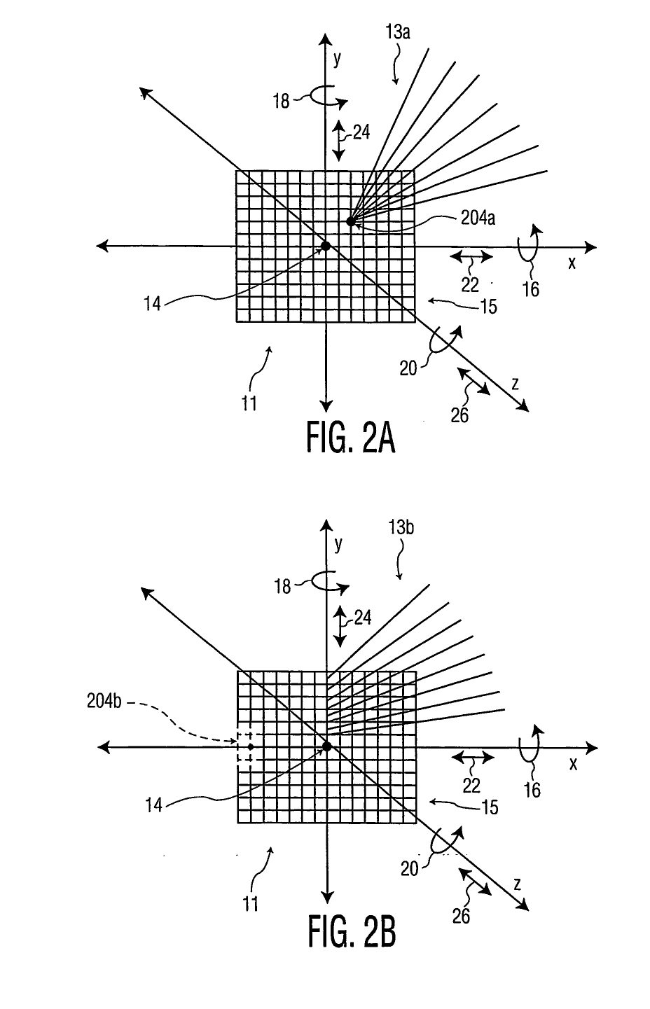

FIGS. 2A-2B are schematic diagrams of slices generated by the 2-D array of FIG. 1;

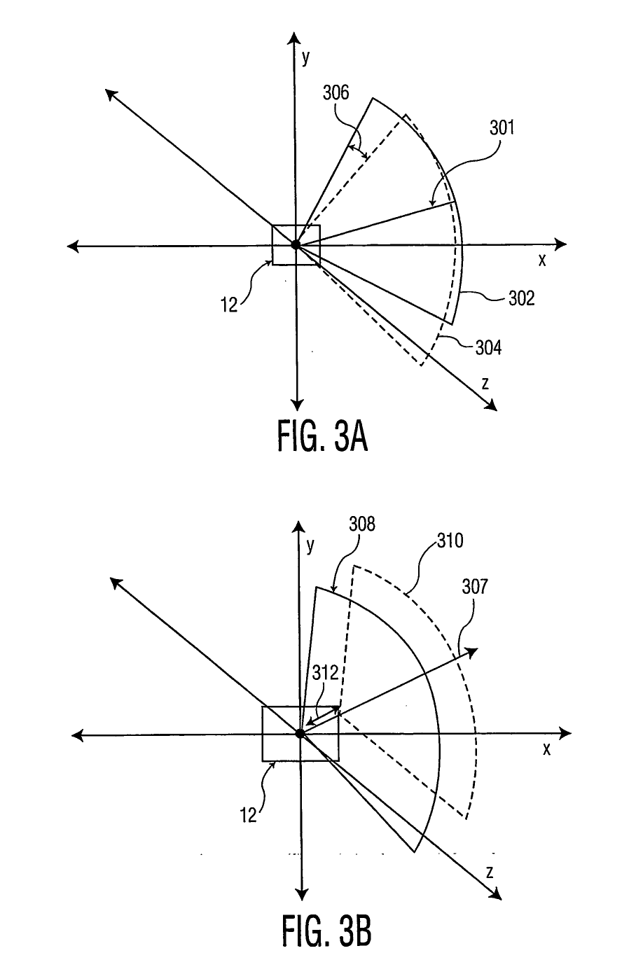

FIG. 3A is a schematic diagram of rotation of a slice in accordance with the present invention;

FIG. 3B is a schematic diagram of translation of a slice in accordance with the present invention; and

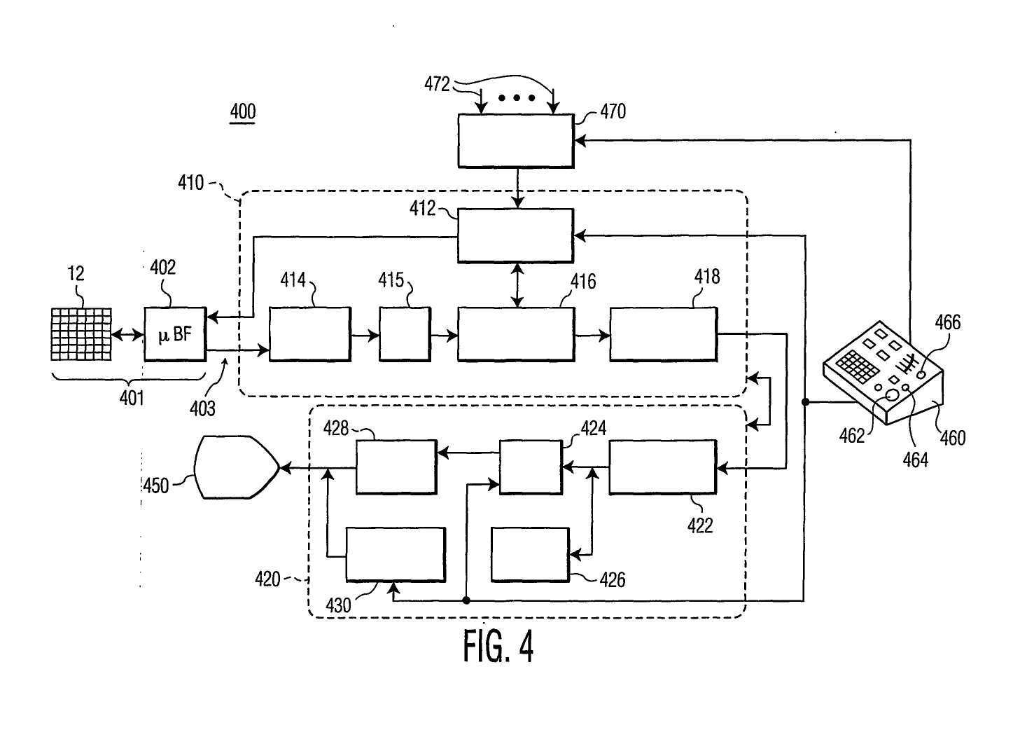

FIG. 4 is a block diagram of an ultrasound system in accordance with the present invention.

An ultrasound system is provided for acquiring a series of images using at least one selectable imaging acquisition parameter such as at least one automatic adjustment parameter, for sequentially acquiring images of the series of images, where sequential acquisitions (or selected acquisitions of the series of acquisitions) are performed and adjusted in accordance with the automatic adjustment parameter for stepping through a sequence of adjustments. The series of images may be acquired sequentially at a slow rate that can be selected to be slow enough for the series of acquired images to be spread out over time, i.e., not substantially simultaneous.

The imaging acquisition parameter can further include at least one of scan line density, frame rate, location of origin of scan lines (apex), trigger for acquiring images, range of stepping parameter (outside limits for performing adjustments), stepping selection for selecting acquisitions for which adjustments are to be performed, color flowbox parameters, etc.

The automatic adjustment parameter can include, but is not limited to, at least one of axis of rotation, rotation angle adjustment factor of rotation, distribution of scan lines about axis of rotation, axis of translation, translation adjustment factor, and adjustment factors for image configuration parameters (e.g., at least one dimension defining a shape of an image or slice being acquired, and the type of shape of the image or slice being acquired, selectable from a predefined list of shapes, e.g., sector, trapezoid, etc.), receive gain, transmit power, receive aperture configuration, receive apodization profile, transmit aperture configuration, transmit apodization profile, etc.

The adjustment factors may be increments, decrements (which is a negative increment), derived from a formula or other mapping, and/or selected from a predefined set or list of selections, and may be herein referred to as increments. The trigger can be associated with at least one event that is timed (synchronous) or dependent on a synchronous event such as a clock pulse or a time delay, or at least one event that is not timed (asynchronous), such as a predetermined point within a heart beat cycle, a predetermined point within a respiration cycle, manual activation of an actuator such as a trigger, foot pedal, button, switch, etc.

With reference to FIG. 1, an exemplary 2-D array 12 of individually controllable elements of a transducer 11 of an ultrasonic probe is shown schematically. While the probe will be referred to as a fully sampled array in the examples below (where each element is individually addressable), a sparse array (where a subset of the physical set of transducer elements is addressable and controllable, or equivalently there is a pattern of physical gaps between some elements such that they are not all contiguous, or the addressable and controllable ones are not all contiguous) configuration is also possible. The probe may be any array having elements distributed in a plurality of dimensions. Furthermore, although the examples provided below are primarily with reference to a 2-D probe, it is envisioned that the probe may be a 1-D array.

The transducer 11 receives ultrasonic energy, typically pulsed energy, from an ultrasonic generator (not shown) and emits a single scan line in response to each pulse. A sequence of scan lines typically forms a sector shape, also known as a sweep. For a 2-D array probe the set of scan lines correspond to a slice 13 which typically lies in a scan plane. Ultrasound data is typically acquired in frames, where each frame represents one or more sweeps. The scan lines are steered by a beamformer assembly (not shown) as described in greater detail below with reference to FIG. 4, so that scan lines are transmitted in various directions through a volumetric region in front of the probe. As successive slices are imaged, each slice is changed, i.e., redirected, relocated or reshaped, in accordance with an incremental change of at least one parameter relative to a previously imaged slice. A large number (a set) of the successive slices slightly displaced in a selected direction or rotated about a selected axis can be used to interrogate a volume. Echo data corresponding to each slice is processed by an image processing assembly (not shown) for generating a 2-D image or rendering a 3D image.

The shape of slice 13 is shown substantially as a sector. Those having ordinary skill in the art will recognize that the slice 13 may have other shapes, such,; as trapezoid, parallelogram, and may have one or more curves or angles, each having a radius, where the slice is not planar.

Each successive slice is changed relative to a previously imaged slice in at least one of; and preferably only one, of rotation about a selected axis by a rotation angle adjustment factor, or translation along a selected axis by a translation adjustment factor. Preferably the adjustment factor value is fixed for a set of successive slices, but is not limited thereto Individual scan lines of a respective slice, and individual slices of a series of slices are preferably changed (e.g., rotated or translated) using the same adjustment factor value, but it is envisioned that different a adjustment may be used for respective scan lines in a slice, or slices in a series of slices, such as by performing a function or algorithm on the respective adjustment factor value used for the slice.

The 2-D array 12 lies in a plane that is defined by the X and Y axes, and the Z axis lies orthogonal to the plane of the 2-D array 12. Each scan line has a point of origin, where a series of scan lines associated with a slice typically, but not limited thereto, shares one point of origin, where the shared point of origin is known as an apex. It is common for the apex to be designated to be at the geographic center of the 2-D array 12, herein referred to as the origin 14, however it is known, and it is often beneficial to designate the apex to be at a different location within acoustic limitations of the transducer, where the apex may lie in front of or behind the transducer face 15 (i.e., be shifted in along the Z axis relative to the origin 14), and/or where the apex location may be shifted along the X and/or Y axes relative to the origin 14.

When a point of origin of a scan line is other than the origin 14, the scan line appears to have a virtual point of emanation (VPE) and/or a virtual point of reception (VPR), herein collectively referred to as a VPE, where the scan line emanates from and received, respectively, by the 2-D array 12. Scan lines of a slice that share a common apex positioned at a location other than at the origin 14 have unique VPEs.

In the example described, a series of slices are imaged, where successive slices are rotated about a selectable axis of rotation. Exemplary rotation about the X, Y and Z axes are shown by arrows 16, 18, 20, respectively. Furthermore, other axes of rotation may be selected, as described further below. Alternatively, a series of slices may be imaged in which successive parallel slices are shifted along a selectable axis of translation, where preferably, but not limited thereto, the plane of each slice remains orthogonal to the axis. Exemplary translation about the X, Y and Z axes are shown by arrows 22, 24, 26, respectively. Translation along a selected axis can be effected by incrementally changing the apex position for successive slices.

The axis of rotation and the axis of translation may be selected from a line in space for which scan lines of the set of scan lines generated during one of rotation about that line or translation along that line still lie within an acoustic field of view (AFOV) of the probe, where the AFOV is the region in space in front of the probe face in which acoustic pulses can be focused during transmit and receive within the practical limitations of the probe geometry and construction, including limitations such as element pitch, aperture width, array frequency, etc. For a 1-D array probe, the AFOV is typically shaped like a triangular sector having its apex behind the probe face. For a 2-D array probe, the AFOV is typically shaped like a conical frustum having its apex behind the probe face. The axis of rotation and axis of translation accordingly may be selected from a line lying within the AFOV of the probe, or alternatively lying outside the AFOV of the probe, such as on the probe face if the probe face is planar or curved, or tangent to the probe face if it is curved. The apex for individual slices or for the series of slices may be selected within the acoustic limitations of the transducer, where the apex is displaced from the origin 14 by selected respective distances in the x, y and z directions.

In FIG. 1 the scan lines of slice 13 emanate from the transducer face 15, where the apex of the scan lines is located at the origin 14. FIG. 2A shows a slice 13A having scan lines emanating from the transducer face 15, where the apex 204a of the scan lines is located directly in front of the origin 14. FIG. 2B shows a slice 13b having scan lines emanating from the transducer face 15, where the apex 204b (shown in phantom) of the scan lines is located behind and to the left of the origin 14. It is further contemplated that a different apex may be selected for respective scan lines.

Rotation of a slice about a selected axis of rotation 301 is shown in FIG. 3A. An apex, shape and orientation for an initial (i.e., first) slice 302 is selected by the user or predefined, where the orientation is selected from the orientation of any plane lying within the AFOV of the probe. An axis of rotation and a rotation angle adjustment factor 306 are further selected, where preferably the axis of rotation is the center scan line of the initial slice 302, however the axis of rotation may be selected to be any scan line of the initial slice 302, or alternatively any line lying within the AFOV of the probe, or from a line in space for which scan lines of the set of scan lines generated during rotation about that line still lie within the AFOV of the probe. For example, an axis of rotation may be selected from within the probe face if it is planar or curved, or tangent to the probe face if it is curved, as in the case of a curved transducer array. The initial slice 302 is imaged, after which a second slice 304 (shown by dotted lines) is imaged by incrementally rotating about the axis of rotation 301, and where the amount of rotation is defined by the rotation angle adjustment factor 306. Subsequent slices (not shown) are imaged by rotating about the axis of rotation 301, where the amount of rotation for each subsequent slice is preferably defined by the same angle adjustment factor 306, however, it is contemplated that the angle adjustment factor may not be fixed and may be calculated in accordance with a function or algorithm Similarly, for a 1-D array probe, the axis of rotation is selected from any line lying within the AFOV of the probe, or from a line in space for which scan lines of the set of scan lines generated during rotation about that line still lie within the AFOV of the probe. Accordingly, the axis of rotation may be selected from a line within the probe face or tangent to it. The orientation of the initial scan line is selected, and subsequently acquired scan lines are rotated about the selected axis of rotation in accordance with a selected rotation angle adjustment factor.

Translation along an axis of translation 307 is shown in FIG. 3B. An apex, shape and orientation for an initial slice 308 are selected, where the orientation is selected from the orientation of any plane lying within the field of view of the probe. An axis of translation 307 and a translation adjustment factor 312 are further selected, where the axis of translation 307 is preferably orthogonal (but not limited thereto) to the initial slice.

The initial slice 308 is imaged, after which a second slice 310 (shown by dotted lines), parallel to the initial slice, is imaged by translating the plane in which the initial slice 308 lies (while maintaining the orientation of the plane (orthogonal) relative to the axis of translation) along the axis of translation 307, where the translated plane defines the plane in which the second slice lies, and where the amount of translation is defined by the translation adjustment factor 312. Subsequent parallel slices (not shown) are imaged by translating as described above, where the amount of translation for each subsequent slice is preferably defined by the same translation adjustment factor 312, however it is contemplated that the translation adjustment factor may not be fixed and may be calculated in accordance with a function or algorithm.

Similarly, for a 1-D array probe, the axis of translation is selected from any line lying within the AFOV of the probe, or from a line in space for which scan lines of the set of scan lines generated during translation along that line still lie within the AFOV of the probe, such as a line in space within or tangent to the probe face. The orientation of the initial scan line is selected, and subsequently acquired scan lines are translated along the selected axis of translation in accordance with a selected translation adjustment factor.

In another embodiment of the invention, the images of the series of images are each a 3-D image, such as a 3-D rendering obtained from a set of 2-D images, where the set of 2-D images may be obtained by automatic adjustment in accordance with the present invention or by another method. Accordingly, a series of sets of 2-D images are acquired. Each set of 2-D images is acquired by automatic adjustment (e.g., incrementation, decrementation (which is negative incrementation), selection from a predefined list, and/or in accordance with a function or other mapping) in accordance with a selectable automatic adjustment parameter. For example, each successive set of 2-D images is acquired by rotation about a selected axis by a rotation angle adjustment factor, or translation along a selected axis by a translation adjustment factor relative to a previously acquired set of 2-D images, such as by rotating or translating the first slice acquired of the rotated or translated set of 2-D images relative to the first slice acquired of the previous set of 2-D images in accordance with the adjustment factor. The axis of rotation and the axis of translation are selectable from any line in the AFOV of the probe, or from a line in space for which scan lines of the set of scan lines generated during one of rotation about that line or translation along that line still lie within the AFOV of the probe, such as a line in space within or tangent to the probe face.

FIG. 4 illustrates in block diagram form an exemplary ultrasound system 400 constructed in accordance with the principles of the present invention. In a preferred embodiment the probe 401 includes the 2-D array of transducer elements 12 and a sub-array processor or micro-beamformer 402. The micro-beamformer 402 contains circuitry which control the signals applied to groups of elements (“patches”) of the 2-D array 12 and does some processing of the echo signals received by elements of each group. Micro-beamforming in the probe, 401 advantageously reduces the number of conductors in a cable 403 between the probe and other components of the ultrasound system 400 and is described in U.S. Pat. No. 5,997,479 (Savord et al.) and in U.S. Pat. No. 6,436,048 (Pesque). The ultrasound system is not limited to use with a micro-beamformer, and other beamforming systems may be used instead. Furthermore, the beamforming system may be configured for beamforming signals transmitted from and/or received by a 1-D probe.

The probe 401 is coupled to an exemplary scanner 410 of the ultrasound system. The scanner 410 includes a beamformer controller 412 which is responsive to at least one user input device 460 and provides control signals to the micro-beamformer 402 and/or beamformer 416 for controlling beamformer parameters including timing, frequency, direction and focusing of transmitted ultrasound beams and beamforming of received echo signals for effecting incremental changes, such as via rotation or translation, of a slice being acquired relative to a previous slice acquired. Received echo signals are provided to the micro-beamformer 402 which performs a portion of the receive beamforming processing and provides the processed signals to the scanner 410.

Within the scanner 410 the processed signals are processed by preamplifier and TGC (time gain control) circuitry 414, and then digitized by the A/D converters 415. The digitized echo signals are then formed into beams by a beamformer 416. The echo signals are next processed by an image processor 418 which performs digital filtering, B mode detection, and Doppler processing, and can also perform other signal processing such as harmonic separation, speckle reduction through frequency compounding, and other desired image processing.

The echo signals processed by the image processor 418 are output by the scanner 410 and provided to an exemplary digital display subsystem 420, which processes the echo signals for display in the desired image format. The echo signals are processed by an image line processor 422, which is capable of sampling the echo signals, splicing segments of beams into complete line signals, and averaging line signals for signal-to-noise improvement or flow persistence. The image lines are scan converted into the desired image format by a scan converter 424 which performs R-theta conversion as is known in the art. The image is then stored in an image memory 428 from which it can be displayed on a display device 450.

The image in memory is also overlaid with graphics to be displayed with the image, which are generated by a graphics generator 430 which is responsive to the user control for the input of patient identifying information or the movement of cursors, for example. The ultrasound image on the display 450 may also be accompanied by one or more icons which depict the position of the initial image plane with respect to the probe and the selected automatic adjustment parameters. Individual images or image sequences can be stored in a cine memory 426 during capture of image loops. Other display subsystems may be used for processing the echo signals, as known in the art. For real-time volumetric imaging the display subsystem 420 also includes a 3D image rendering processor (not shown) which receives image lines from the image line processor 422 for the rendering of a real-time three dimensional image which is displayed on the display device 450.

In accordance with the principles of the present invention, the at least one user input device 460 includes controls 462-466 for allowing a user to make user selections including initialization parameters, such as the location of an apex (apex location parameter), image or slice configuration (including type of shape selectable from a predefined list of shapes and one or more dimensions defining the shape) of an initial slice (initial image configuration parameter), plane orientation (tilt) for a first (initial) slice (plane orientation parameter), where orientation refers to orientation of a plane of the initial slice selectable from any plane lying within the field of view of the probe, scan line rate, scan line density, frame rate, trigger conditions for acquiring respective images, initial gain, and/or initial power, etc.

User selections may further be made for at least one automatic adjustment parameter, such as an axis of rotation selectable from a line in space for which scan lines of the set of scan lines generated during rotation about that line still lie within the AFOV of the probe, and a rotation angle adjustment factor (i.e., the angle at which respective successive slices are rotated about the axis of rotation), an axis of translation selectable from a line in space for which scan lines of the set of scan lines generated during translation along that line still lie within the AFOV of the probe, and with the axis of translation at a predetermined angle (preferably orthogonal) to the plane of the first slice, and a translation adjustment factor (i.e., the amount respective successive slices are translated along the axis of translation while maintaining a predetermined angle with respect to the axis of translation).

Additional adjustment parameters may include a receive gain adjustment factor (i.e., the amount gain is increased or decreased for respective adjustments), a transmit power adjustment factor (i.e., the amount transmit power is increased or decreased for respective adjustments), respective receive and transmit aperture configuration adjustment factors (e.g., predetermined individual or groups of elements designated for activation thereof), respective receive and transmit apodization gain profile adjustment factors (for determining configuration of the gain profile, e.g., selecting from a list of shapes, such as circular Hamming, cylindrical, rectangular, cubic, etc.; degree of flatness of the profile, etc., range of adjustment (e.g., outside limits for incrementation or decrementation), and/or image configuration parameter adjustment factors, including incrementation or decrementation of one or more dimensions defining a shape of a slice being acquired and/or selection of type of shape of the slice being acquired from a predetermined list of types of shapes, e.g., sector, trapezoid, etc. In addition, user selections may include option parameters such as flow box parameters, including size and location of a flowbox in the initial slice.

As the user manipulates these controls, signals from the controls are coupled to the beamformer controller 412. The beamformer controller 412 responds to the user's initialization parameter selections for the initial slice by adjusting at least one beamforming parameter by programming the sequence of beams to be transmitted and received by the beamformer 416 and/or the micro-beamformer 402 in a frame table. The beamformer controller 412 programs a frame table for each image by recalculating or selecting the proper sequence of focusing coefficients and/or aperture selection for activation of groups of transducer elements for transmit and receive beamforming for focusing and steering the ultrasound beams in the directions needed to scan the initial slice and successive slices in accordance with the initialization parameters specified by the user.

The transmit beams are transmitted, delayed and focused in the desired directions through the AFOV of the probe under control of a transmit beamformer in the micro-beamformer 402 and/or the beamformer 416. Preferably a frame table is pre-calculated or calculated on the fly to contain data for controlling the micro-beamformer 402 and or the'beamformer 416 for achieving transmission and reception of the beams necessary to produce the desired initial slice and subsequent slices in accordance with the user selections.

When user selections include a flowbox, the flowbox is recreated in a corresponding position in each successively obtained image. Alternatively, the position of the flowbox may be changed in successively obtained images according to the automatic adjustment parameter. Scan lines located in the flowbox are repetitively generated in groups, each scan line group containing scan lines that are steered in the same direction. The groups are referred to as Doppler packets, and the receive echoes are recorded as Doppler ensembles, as is well understood in the art. B mode echoes from each received scan line both outside and throughout the flowbox are processed by amplitude detection in the image processor 418. Doppler echo ensembles from scan lines within the flowbox are Doppler processed in the image line processor 422 for the production of display signals depicting flow or tissue motion. The processed B mode and/or Doppler signals are then coupled to the display subsystem 420 for display.

The at least one user input device 460 is preferably further coupled to the, display subsystem 420, where the scan converter 424 and the graphics generator 430 are informed of the design of the images. This enables the scan converter to anticipate and then properly locate the Doppler information along the scan lines of a specified color box area, and enables the graphics generator to outline or highlight the color box if desired. The final image is then displayed on the display device 450.

The beamformer controller 412 includes a processing assembly such as at least one processor, microprocessor, CPU, integrated circuit, ASIC, FPGA, networking circuitry and/or logic circuitry for executing programmable instructions for processing the user input parameters and controlling the micro-beamformer 402 and/or the beamformer 416, including adjusting the beamformer parameters, such as by creating the frame table. The executable instructions may be provided as at least one software module executable by the processing assembly, where the at least one software module may be stored on a computer readable medium accessible by the processing assembly, and/or propagated via a transmission medium to the processing assembly.

A trigger controller 470 receiving user input trigger parameters from the at least one user input device 460 and at least one trigger signal 472 is coupled to the beamformer controller 412 for controlling the timing of control signals sent by the beamformer controller 412 to the micro-beamformer 402 and/or the beamformer 416, where the control signals implement adjustment of the desired automatic adjustment parameter. Trigger signal types include, for example, at least one user actuator signal, a respiration cycle signal, a cardiac cycle signal, and/or a clock signal. The trigger parameter instructs the trigger controller 470 to control the beamformer controller 412 to process received trigger signals that correspond to a selected trigger signal type or a selected combination of trigger signal types. The trigger parameter may include instructions for controlling at least one of the trigger controller 470 and the beamformer controller 412 to perform adjustments at specified intervals, where an interval may occur with each trigger signal or at specified trigger signals, and where the intervals may be regular, irregular, in accordance with a function or in accordance with specified conditions, etc.

Received trigger signals 472 are processed in accordance with the trigger parameters for determining when to perform an adjustment. Upon determining that an adjustment is to be performed, the adjustment is processed in accordance with the at least one adjustment parameter, and a next image of the series of images is acquired. Accordingly, a volume can be interrogated over a relatively short period of time or an extended period of time by automatically incrementally steering ultrasound beams through the volume by rotation or translation and acquiring an image after each incremental change, where each incremental change is performed in response to an occurrence of a selected event, where the events may occur synchronously or asynchronously. The trigger controller 470 includes at least one processor and/or logic circuitry, such as a multiplexer. The at least one processor includes at least one processor, microprocessor, CPU, integrated circuit, ASIC and/or networking circuitry for executing programmable instructions. The executable instructions may be provided as at least one software module executable by the at least one processor, where the at least one software module may be stored on a computer readable medium accessible by the at least one processor, and/or propagated via a transmission medium to the at least one processor.

Actuator signals may be generated by a user operated button, foot pedal, switch, etc., for causing an adjustment to be processed and an image to be acquired when in conjunction with user actuation. The clock signal is generated by a clock that may be included in the trigger controller 470, the beamformer controller 412, the probe 401, a clock external to the ultrasound system, a clock associated with another processor of the ultrasound system, etc.

The cardiac cycles signal are generated by an electrocardiograph generator in accordance with an EKG signal, for triggering the ultrasound system to process an adjustment and acquire an image when the cardiac cycle reaches a particular stage, so that the series of 2-D images may be associated with one or more particular stages of the cardiac cycle, and measurements obtained from processing the series of 2-D images correspond to the desired stage of the cardiac cycle. The respiratory cycle signals are generated by a respiratory gating device for triggering the ultrasound system to process an adjustment and acquire an image when the respiratory cycle reaches a particular stage, so that the series of 2-D images may be associated with one or more particular stages of the respiratory cycle, and measurements obtained from processing the series of 2-D images correspond to the desired stage of the respiratory cycle.

The series of 2-D images may be further processed for generating a 3-D rendering of the imaged volume, for live viewing, or for storage in cine memory for later cine viewing at variable replay speeds. Furthermore, the series of 2-D images may be processed for displaying the volume imaged using the known technique of display persistence, in which corresponding pixel data (e.g., intensity) of subsequent 2-D images are averaged, and the resulting average intensity is displayed at the corresponding pixel location, for obtaining a displayable volume image with minimal processing.

By imaging a series of 2-D images, which are automatically rotated in accordance with the present invention, of a generally spherical structure, such as the heart or a chamber thereof, where the axis of rotation is aligned with a central axis of the structure, using display persistence a live or stored high quality approximation of a displayable 3-D volume projection is obtainable with minimal processing. Displayed volume images may be further processed, such as for acoustic quantification (AQ), border detection, and/or obtaining volume measurements, such as by using the method of disks. The data obtained may further be used, such as for examining cardiac efficiency, such as for determining Ejection Fraction and obtaining stress measurements.

During an imaging procedure, the probe of the ultrasound system is generally coupled to the body of a patient by an acoustic couplant (a gel) which is spread over the slin of the patient. A good acoustic window is established when the image plane (i.e., the plane of a slice being imaged) images the interior of the body without obstruction or substantial attenuation of the ultrasonic waves. The probe may be held in place, maintaining the favorable acoustic window, while the image plane is automatically rotated or translated in accordance with user entered user selections and trigger signals received. In this way the orientation of the image plane is adjusted while maintaining a favorable acoustic window with the body. The advantage of this is that the user need not manipulate system controls while concentrating on maintaining the favorable acoustic window, because the system automatically adjusts the parameter(s) chosen by the user for stepping through a volume.

The present invention has particular utility for telemedicine, when a skilled diagnostician is not present at the patient's location. For instance, a medic can hold the ultrasound probe against the body of an accident victim who is suspected to have internal injuries or bleeding. The image data can be radioed or otherwise communicated to a facility having at least one user input device 460, a beamformer controller 412, a display subsystem 420 and a display device 450, where the image is displayed for a skilled diagnostician.

The diagnostician can manipulate the user controls at his location for entering initialization parameters and automatic adjustment parameters, with control signals from the beamformer controller 412 being communicated back to the beamformer 416 and/or micro-beamformer 402 of the portion of the ultrasound system at the site of the accident. The orientation of the initial slice and successive slices is adjusted correspondingly by the skilled diagnostician. While the medic holds the probe stationary against the accident victim, the diagnostician can manipulate the image plane remotely for automatically obtaining a series of 2-D images to survey the suspected injury area and recommend treatment from the remote location. An ultrasound system by which the user controls can be operated remotely for such a procedure is described in U.S. Pat. No 5,715,823.

While the present invention finds great utility when embodied in transthoracic and other probes which are intended to be used from outside the body, indwelling probes may also benefit from the present invention. For example, a TEE probe may be fabricated with the ability to automatically steer the plane orientation as described above. Multiplane (omniplane) TEE probes provide the ability to reorient the image plane by moving the probe up and down in the esophagus, twisting the insertion tube in the esophagus, articulating the probe tip, and rotating the array transducer. However, even greater versatility is obtainable by providing the degrees of freedom of automatic image plane orientation for a series of images of the present invention, which can also obviate the need for some of the mechanical plane adjustments presently needed for TEE probes. Although the user controls in the embodiment of FIG. 4 are shown located on an ultrasound system user interface device, it will be appreciated that the user controls may also be located on the probe. This would enable the user to manipulate the image plane orientation from the probe, without the need to access the ultrasound system scanner or cart.

It will be understood that various modifications may be made to the embodiments disclosed herein. Therefore, the above description should not be construed as limiting, but merely as exemplifications of preferred embodiments. Those skilled in the art will envision other modifications within the scope and spirit of the claims appended hereto.

1. An ultrasound imaging system (400) for acquiring a series of images, the ultrasound system (400) comprising:
a transducer (11) for emitting and receiving ultrasound energy;
a beamformer assembly (402) for beamforming in accordance with at least one beamforming parameter the emitted and received ultrasound energy for generating a plurality of scan lines and acquiring a series of images;
at least one user input device (460) for enabling a user to select at least one automatic adjustment parameter including an axis selectable from a line in space for which scan lines of a set of scan lines generated during one of rotation about the line in space or translation along the line in space lie within an acoustic field of view of the transducer (11), and an adjustment factor; and
a control unit (412) for controlling the beamformer assembly (402) during acquisition of the series of images for adjusting the at least one beamformer parameter for an image being acquired with respect to a previous image acquired in accordance with the at least one automatic adjustment parameter, for at least one of rotating about the axis and translating along the axis the image being acquired with respect to the previous image by an amount defined by the adjustment factor.

2. The ultrasound imaging system according to claim 1,
wherein the ultrasound imaging system (400) further comprises circuitry for receiving a plurality of trigger signals (472) driven by the occurrence of an event, including at least one trigger signal driven by at least an asynchronous event; and
wherein the control unit (412) acquires individual images of the series of images in response to receipt of a respective at least one trigger signal of the at least one trigger signal.

3. The ultrasound imaging system (400) according to claim 1,
wherein the transducer (11) includes an array of transducer elements; and
wherein an apex location is further selectable via the at least one user input device (460), wherein the apex location is selectable from at least a point other than the geographic center of the array of transducer elements, and wherein the selected apex location determines an apex of at least a portion of the plurality of scan lines corresponding to the first image of the series of images.

4. The ultrasound imaging system (400) according to claim 1, wherein each image of the series of images is a 2-D image.

5. The ultrasound imaging system (400) according to claim 4, wherein at least one plane orientation parameter is further selectable via the user input device (460), wherein the plane orientation parameter is selectable from any plane lying within the acoustic field of view of the transducer (11), and wherein the plane orientation parameter determines orientation of a plane of the first image of the series of images.

6. The ultrasound imaging system (400) according to claim 1, wherein each image of the series of images is a 3-D image.

7. The ultrasound imaging system (400) according to claim 1, wherein the at least one automatic adjustment parameter includes at least one of a gain adjustment factor, a power adjustment factor, an image configuration adjustment factor, a receive aperture adjustment factor, a receive apodization adjustment factor, a transmit apodization adjustment factor, and a transmit aperture adjustment factor, and adjusting the at least one beamformer parameter includes adjusting for acquisition of the image being acquired with respect to acquisition of the previous image at least one of receive gain, transmit power, at least one image configuration parameter, including at least one of type of shape of the image being acquired and at least one dimension defining the shape of the image being acquired, a receive aperture configuration, receive apodization gain profile, transmit apodization gain profile, and a transmit aperture configuration in accordance with the gain adjustment factor, the power adjustment factor, the image configuration adjustment factor, the receive aperture adjustment factor, the receive apodization adjustment factor, the transmit apodization adjustment factor, and the transmit aperture adjustment factor, respectively.

8. The ultrasound imaging system (400) according to claim 2, wherein the at least one trigger signal includes at least one of at least one actuator signal generated by a user operated actuator, at least one cardiac cycle signal generated by an electro-cardiograph generator, and at least one respiration cycle signal generated by a respiratory gating device.

9. The ultrasound imaging system (400) according to claim 1, wherein the series of images are processed for being displayed on a display device (450) using display persistence.

10. The ultrasound imaging system (400) according to claim 1, wherein the transducer (11) includes an array having elements distributed in a plurality of dimensions.

11. An ultrasound imaging system (400) for acquiring a series of images, the ultrasound system (400) comprising:
a transducer (11) for emitting and receiving ultrasound energy;
a beamformer assembly (402) for beamforming in accordance with at least one beamforming parameter the emitted and received ultrasound energy for acquiring a series of images;
circuitry for receiving a plurality of trigger signals (472) driven by the occurrence of an event including at least one trigger signal driven by at least an asynchronous event; and
a control unit (412) for controlling the beamformer assembly (402) during acquisition of the series of images for providing for acquiring individual images of the series of images in accordance with receipt of a respective at least one trigger signal of the at least one trigger signal, wherein the controlling includes adjusting the at least one beamformer parameter for an image acquisition with respect to a previous image acquisition in accordance with at least one predetermined automatic adjustment parameter, wherein the adjusting includes providing for shifting positioning of the image being acquired with respect to the previous image acquisition.

12. The ultrasound imaging system (400) according to claim 11, wherein the at least one trigger signal includes at least one of at least one actuator signal generated by a user operated actuator, at least one cardiac cycle signal generated by an electro-cardiograph generator, and at least one respiration cycle signal generated by a respiratory gating device.

13. The ultrasound imaging system (400) according to claim 11, wherein the beamformer assembly (402) further generates a plurality of scan lines for acquiring the series of images, and wherein the automatic adjustment parameter includes an axis selectable from a line in space for which scan lines of a set of scan lines generated during one of rotation about the line in space or translation along the line in space lie within an acoustic field of view of the transducer (11) and an adjustment factor, and adjusting the at least one beamformer parameter includes at least one of rotating about the axis and translating along the axis the image being acquired with respect to the previous image by an amount defined by the adjustment factor.

14. The ultrasound imaging system (400) according to claim 11, wherein each image of the series of images is a 2-D image.

15. The ultrasound imaging system (400) according to claim 14, wherein orientation of a plane of the first image of the series of images is determined in accordance with a predefined plane orientation parameter selectable from any plane lying within the acoustical field of view of the transducer (11).

16. The ultrasound imaging system (400) according to claim 11, wherein each image of the series of images is a 3-D image.

17. The ultrasound imaging system (400) according to claim 11, wherein the at least one automatic adjustment parameter includes at least one of a gain adjustment factor, a power adjustment factor, an image configuration adjustment factor, a receive aperture adjustment factor, a receive apodization adjustment factor, a transmit apodization adjustment factor, and a transmit aperture adjustment factor, and adjusting the at least one beamformer parameter includes adjusting for acquisition of the image being acquired with respect to acquisition of the previous image at least one of receive gain, transmit power, at least one image configuration parameter, including at least one of type of shape of the image being acquired and at least one dimension defining the shape of the image being acquired, a receive aperture configuration, receive apodization gain profile, transmit apodization gain profile, and a transmit aperture configuration in accordance with the gain adjustment factor, the power adjustment factor, the image configuration adjustment factor, the receive aperture adjustment factor, the receive apodization adjustment factor, the transmit apodization adjustment factor, and the transmit aperture adjustment factor, respectively.

18. The ultrasound imaging system (400) according to claim 11, wherein the providing for shifting positioning includes providing for at least one of translating, rotating and changing the shape of the image being acquired with respect to a previous image acquisition.

19. The ultrasound imaging system (400) according to claim 11, wherein the transducer (11) includes an array having elements distributed in a plurality of dimensions.

20. A method of ultrasonically imaging with a transducer array for generating a plurality of scan lines for imaging a region of interest of a body comprising the steps of:
providing for receiving at least one automatic adjustment parameter including an axis selectable from a line in space for which scan lines of a set of scan lines generated during one of rotation about the line in space or translation along the line in space lie within an acoustic field of view of the transducer array and an adjustment factor; and
providing for controlling beamforming of at least one of ultrasound energy emitted by the transducer array and ultrasound energy received by the transducer array in accordance with at least one beamforming parameter for acquiring a series of images by adjusting the at least one beamformer parameter for an image being acquired with respect to a previous image acquired in accordance with the at least one automatic adjustment parameter for at least one of rotating about the axis and translating along the axis the image being acquired with respect to the previous image by an amount defined by the adjustment factor.

21. The method according to claim 20, further comprising the step of receiving a plurality of trigger signals including at least one trigger signal driven by at least an asynchronous event, wherein the controlling step further includes acquiring respective images of the series of images in response to receipt of a respective at least one trigger signal of the plurality of trigger signals.

22. A method of ultrasonically imaging with a transducer array a region of interest of a body comprising the steps of:
providing for receiving at least one automatic adjustment parameter;
providing for receiving a plurality of trigger signals driven by an event including at least one trigger signal driven by at least an asynchronous event; and
providing for controlling beamforming of at least one of ultrasound energy emitted by the transducer array and ultrasound energy received by the transducer array in accordance with at least one beamforming parameter for acquiring a series of images, where respective images of the series of images are acquired in response to receipt of a respective at least one trigger signal of the at least one trigger signal, including adjusting the at least one beamformer parameter for an image acquisition with respect to a previous image acquisition in accordance with the at least one automatic adjustment parameter, wherein the adjusting includes providing for shifting positioning of the image being acquired with respect to the previous image acquisition.

23. The method according to claim 22, wherein the at least one trigger signal includes at least one of at least one actuator signal generated by a user operated actuator, at least one cardiac cycle signal generated by an electro-cardiographic generator, and at least one respiration cycle signal generated by a respiratory gating device.

24. The method according to claim 22, wherein the providing for shifting positioning includes at least one of providing for translating, rotating and changing the shape of the image being acquired with respect to a previous image acquisition.
